(19) United States
(12) Patent Application Publication
(10) Pub. No.: US 2020/0030342 A1
(43) Pub. Date: Jan. 30, 2020

(54) DEHYDROEPIANDROSTERONE (DHEA) SUPPLEMENTATION ON FEMALE SEXUAL DESIRE AND FUNCTION IN PRE-MENOPAUSAL WOMEN

(71) Applicants: Norbert Gleicher, (US); Vitaly Kushnir, (US); David H. Barad, (US); American Infertility of New York, P.C., New York, NY (US)

(72) Inventors: Norbert Gleicher, New York, NY (US); Vitaly A. Kushnir, New York, NY (US); David H. Barad, Closter, NJ (US)

(73) Assignees: American Infertility of New York, P.C., New York, NY (US); Norbert Gleicher, New York, NY (US); Vitaly A. Kushnir, New York, NY (US); David H. Barad, New York, NY (US)

(21) Appl. No.: 16/048,004

(22) Filed: Jul. 27, 2018

(51) Int. Cl. A61K 31/566; A61P 5/00; G16H 10/60; G16H 10/20

(52) U.S. Cl. CPC A61K 31/566; G16H 10/20; G16H 10/60; A61P 5/00

(57) ABSTRACT

A method of improving sexual function in premenopausal women, who have low Female Sexual Function Index (FSFI) baseline of less than or equal to 25.7. The method includes providing information to premenopausal women to provide input for calculating the FSFI score and, if the score is low, providing dehydroepiandrosterone (DHEA) supplementation to be taken in 25 mg dosages daily once, twice, thrice or four times over a period of time of four to six weeks. By the end of the period of time, the FSFI baselines of the premenopausal women improves by at least seven percent, serum androgen levels of the premenopausal women increase and follicle-stimulating hormone (FSH) levels of the premenopausal women decrease.

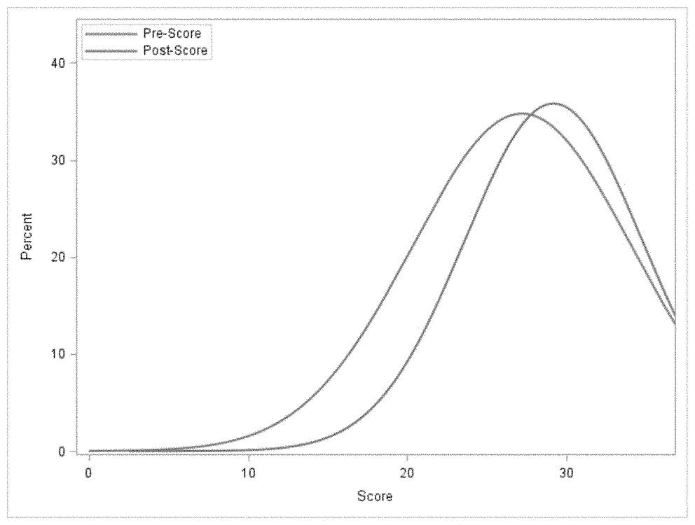

### CROSS-REFERENCE TO RELATED APPLICATIONS

Not applicable.

### STATEMENT REGARDING FEDERALLY SPONSORED RESEARCH OR DEVELOPMENT

Not applicable.

### THE NAMES OF THE PARTIES TO A JOINT RESEARCH AGREEMENT

Not applicable.

### INCORPORATION-BY-REFERENCE OF MATERIAT SUBMITTED ON A COMPACT DISC OR AS A TEXT FILE VIA THE OFFICE ELECTRONIC FILING SYSTEM (EFS-WEB)

Not applicable.

### STATEMENT REGARDING PRIOR DISCLOSURES BY THE INVENTOR OR A JOINT INVENTOR

Not applicable.

### BACKGROUND OF THE INVENTION

### (1) Field of the Invention

The invention pertains to supplementation with dehydroepiandrosterone (DHEA) to improve sexual desire and function in premenopausal women with low sexual desire and function, objectively assessed by the Female Sexual Function Index (FSFI) score.

### (2) Discussion of Related Art

Poor female sexual desire and sexual dysfunction (FSD) represents significant worldwide problems. In contrast to male sexual dysfunction, FSD is either not addressed, or insufficiently and unsatisfactorily addressed by currently available treatments.² Only one drug, flibanserin, has been approved by the United States Food and Drug Administration (FDA), despite what has been noted as “small treatment effects and substantial safety concerns”.³

Causes of FSD include anatomic, psychogenic, organic etiologies which cause personal distress due to abnormally low desire, arousal, orgasm, or sexual pain.⁴ FSD is often age-related and associated with low serum androgen levels in both pre- and post-menopausal women.⁵⁻⁷ Low androgen levels are associated with female sexual function index domains,⁶ of which, low sexual desire is the most common in women at midlife.⁸ Yet, low sexual desire and sexuality have not been adequately studied.

In healthy women, androgen levels peak during late teens and early twenties, coinciding with peak fertility, and then gradually decline with advancing age. ^{9, 10} Abnormally low androgen levels are often found in women with premature ovarian aging/occult primary ovarian insufficiency (POA/oPOI), in association with aging-related physiologic diminished ovarian reserve (DOR) and in relatively rare cases of adrenal insufficiency.^{11,12} Low androgen levels can also be iatrogenic, as in patients treated with aromatase inhibitors. Such women have also been reported to suffer from low sexual desire.¹³

Low androgen levels are also a common finding among especially older premenopausal women.⁵⁻⁷ Since the literature supports an important role for androgens in female sexual function, it is not surprising that transdermal testosterone (T) supplementation has been used with some success to treat FSD and in particular hypoactive sexual desire disorder (HSDD).^{14, 15} However, the use of systemic dehydroepiandrosterone (DHEA) therapy has been more controversial^{14, 16, 17} and, until now, only demonstrated a marginal degree of effectiveness in improving sexual function in only peri- and post-menopausal women¹⁸ and, possibly, those suffering from adrenal insufficiency.¹⁹ For example, in menopausal women, vaginal administration of DHEA has been found effective and safe in alleviating genitourinary symptoms²⁰, and, therefore, recently received FDA approval for this indication.

Therefore, to the extent DHEA administration has been studied for treatment of F SD, the study has been limited to treatment of pain and/or discomfort during sex, such as is caused by vaginal dryness, pelvic discomfort and pain during intercourse. And, even then, the studies are limited to menopausal women. There have been no studies of sexual function, including libido, desire, arousal and sexual stimuli, particularly for pre-menopausal women.

Particularly for pre-menopausal women, the focus for treatment of FDS has been on discomfort and pain during intercourse such as is caused by vaginal dryness and pelvic discomfort. Yet, female sexuality, arousal and desire have not been studied. There is, therefore, a strong need to provide methods and treatment to increase sex drives, libido, desire and arousal for all women and, particularly, pre-menopausal women who sexually are more active than postmenopausal women.

For over a decade, the Center for Human Reproduction has worldwide pioneered the use of DHEA supplementation in infertile women with POA/oPOI and DOR who have low androgen levels.²¹⁻²⁴ In these women, restoration of adequate androgen levels within the ovarian microenvironment improves ovarian function and fertility treatment outcomes. These positive results are typically the consequence of androgen effects on small growing follicles, mediated via testosterone effects on the androgen-receptors in granulosa cells.²⁵⁻²⁸

In the process of treating thousands of so-affected infertile women, it was documented that many women spontaneously reported improvements in libido, sexual desire and, sometimes, even pain status, leading to the paradoxical situation of women refusing to discontinue DHEA supplementation once they conceived. Yet, a review of the literature provides no studies that investigated the effectiveness of DHEA on female sexuality in premenopausal women.

Therefore, the present inventors undertook a study to so evaluate DHEA supplementation in premenopausal women. In particular, the objective of this study was to investigate the effects of DHEA supplementation on female sexual desire and function in pre-menopausal women. The results confirm that especially pre-menopausal women with objectively low sex drive exhibited improved sex drive—that is, sexual functions of desire, arousal and/or sexuality—after DHEA treatment. Women with the lowest sex drives, exhibited the most pronounced improvements.

The study shows that DHEA treatment for pre-menopausal women stimulates sexual function and sexuality. The result is to do stimulating arousal, desire or libido, rather than, as found in the past, due to treatment of symptoms of painful intercourse such as vaginal dryness and pelvic discomfort. The study shows that, for pre-menopausal women with below normal to very low sexual function, DHEA improves female sex drive.

It would therefore is desirable to provide DHEA supplementation to improve sexual function (or sex drive) in premenopausal women with low baseline FSFI scores.

### SUMMARY OF THE INVENTION

One aspect of the invention resides in DHEA supplementation to improve sexual function (or sex drive) in premenopausal women with low baseline FSFI scores such as those whose baseline FSFI scores are less than 25.7. Preferably, the DHEA supplementation entails a total daily use of micronized DHEA 25 mg up to 100 mg (which is typically split into 25 mg once, twice, three or four times daily).

Another aspect of the invention resides in determining baseline FSFI scores of premenopausal women and, providing the determine baseline FSFI is sufficiently low such as less than 25.7, prescribing or administering DHEA supplementation to improve sexual function (or sex drive). Preferably, the DHEA supplementation entails a total daily use of micronized DHEA 25 mg up to 100 mg (which is typically split into 25 mg once, twice, three or four times daily).

A further aspect of the invention is to realize, after taking the DHEA supplementation in dosages of 25 mg up to 100 mg three times daily for 4-6 weeks, all serum androgen levels increase (each P<0.0001), while FSH levels decrease by 2.6±4.4 from a baseline of 10.3±5.4 mIU/mL (P=0.009). Further, the FSFI score increases by at least 7% (from 27.2±6.9 to 29.2±5.6; P=0.0166). In addition, domain scores for desire increase by 17% (P=0.0004) and by 12% for arousal (P=0.0122). Also, lubrication demonstrates an 8% trend towards improvement (P=0.0551), while no observed changes in domain scores for orgasm, satisfaction or pain. Women having a starting FSFI score of <25.7 experience a 6.1±8.0 (34%) increase in total FSFI score following DHEA supplementation. Among these women, improvements in domain categories are realized for desire (40%), arousal (46%), lubrication (33%), orgasm (54%), satisfaction (24%), and pain (25%).

### BRIEF DESCRIPTION OF THE DRAWING

For a better understanding of the present invention, reference is made to the following description and accompanying drawings, while the scope of the invention is set forth in the appended claims.

The drawing is a distribution diagram that shows the distribution of FSFI scores pre- and post-DHEA supplementation in a group of 50 premenopausal infertile women.

### DETAILED DESCRIPTION OF THE INVENTION

An observational study was conducted in an academically affiliated private fertility center. Patients included 87 premenopausal infertile women, 50 of whom completed the study including the Female Sexual Function Index (FSFI) questionnaires and comprehensive endocrine evaluation before and 4-8 weeks after initiating 25 mg of oral micronized DHEA TID. An example of a FSFI questionnaire is in a FSFI Scoring Appendix at the end of this patent application together with a discussion as to the manner of determining FSFI domain scores and full-scale score, which establish FSFI baselines.

This study received Institutional Review Board (IRB) approval (ER11052015-01) on Nov. 11, 2015 from the IRB of the Center for Human Reproduction. The Center for Human Reproduction is a fertility center that serves a population of infertile women with a high prevalence of low ovarian reserve (LOR), mostly due to advanced female age and/or POA/oPOI among younger women. Since it has been demonstrated that relative hypoandrogenism is a common finding in most infertile patients with LOR^{11, 22, 32, 33}, such patients are routinely pre-supplemented with DHEA based on their androgen levels for a minimum of 6 weeks prior to initiating infertility treatments.

This study was not performed in women who presented for medical care because of sexual dysfunction. To the contrary, the investigated patients in the study uniformly had presented because of longstanding infertility, that is, over 90% had failed prior infertility treatments. Indeed, until they completed a first baseline questionnaire after consenting to participate in this study, the issue of sexual dysfunction was not raised, unless patients themselves brought up the subject during initial consultation, which was almost never the case. Here presented study, therefore, involves premenopausal women with infertility but not women previously diagnosed with FSD. Though it has been suggested that sexual dysfunction may be somewhat more frequent in infertility patients than in the general population^{29, 30} here presented study population approximates much more a general, premenopausal female patient population than one with diagnosis of FSD.

All patients in the study were asked to complete the self-reported Female Sexual Function Index (FSFI) questionnaire³¹ prior to initiation to DHEA supplements and again 4-8 weeks later. The patients' fertility treatments were not affected by their participation in this study, in laboratory investigations, and/or timing of procedures. Patients underwent the same standard endocrine evaluations all patients undergo at the Center for Human Reproduction, including measurement of serum androgen levels: DHEA, DHEAS, total (TT) and free testosterone (FT) before, and 4-8 weeks after, initiating 25 mg of oral DHEA supplementation TID. All here presented hormone levels were obtained by commercial assays (LabCorp, Burlington, N.C.). All androgen levels were measured using high-pressure liquid chromatography/tandem mass spectrometry.

In the study, patients received oral micronized DHEA 25 mg three times daily. However, the total daily use of micronized DHEA of 25 mg up to 100 mg (which is typically split into 25 mg once, twice, three or four times daily) provides desired results.

Fifty out of initial 87 patients (57.5%) who had completed a first evaluation, also submitted within 4-8 weeks the follow-up FSFI questionnaire and had a repeat endocrine evaluation. The 37 patients who did not return for follow-up were considered to have withdrawn from the study if at least two attempts by staff to have them complete a second questionnaire had failed. Most of these patients either chose not to pursue treatment at our center or conceived spontaneously while on DHEA supplementation.

Power analysis indicated that to detect a change in FSFI score of 2.0 with 0.8 power, a total of 50 patients would need to complete a questionnaire following DHEA supplementation. Pearson correlation was used to investigate relationships between FSFI scores and patient demographics, as well as, hormone levels. Comparisons of FSFI scores and endocrine parameters before—and after—DHEA supplementation were made using paired T-tests. Based on starting FSFI scores patients were further subdivided into quartiles. Patient responses before and after DHEA supplementation from those in lowest and highest quartiles were then compared. Groups were compared using a two-sampled T-test or chi-square test. A P-value <0.05 was considered significant. All analyses were conducted using SAS version 9.4 (SAS Institute Inc. Cary, N.C.).

Age of patients was 41.1±4.2 years, BMI 24.4±6.1 kg/m², 86% were married, and 42% were parous. Following supplementation with DHEA, all serum androgen levels increased (each P<0.0001), while FSH levels decreased by 2.6±4.4 from a baseline of 10.3±5.4 mIU/mL (P=0.009). The FSFI score for the whole study group increased by 7% (from 27.2±6.9 to 29.2±5.6; P=0.0166). Domain scores for desire increased by 17% (P=0.0004) and by 12% for arousal (P=0.0122); lubrication demonstrated an 8% trend towards improvement (P=0.0551), while no changes in domain scores for orgasm, satisfaction or pain were observed. Women in the lowest starting FSFI score quartile (<25.7), experienced a 6.1±8.0 (34%) increase in total FSFI score following DHEA supplementation. Among these women improvements in domain categories were noted for desire (40%), arousal (46%), lubrication (33%), orgasm (54%), satisfaction (24%), and pain (25%).

Table 1 shows demographics and baseline endocrine parameters (baseline hormone levels) for 50 patients who completed the study. Table 1 also confirms that 37 women who withdrew from the study following initial evaluation and prior to completing the second FSFI questionnaire had similar clinical characteristics as those who completed the study. Baseline FSFI scores for patients that completed the study were (27.2±6.9; range 2.8-34.8). Pearson correlation showed no relationship between baseline FSFI scores and patient age, body-mass index (BMI), ovarian reserve parameters, anti-Mullerian hormone (AMH) and follicle stimulating hormone (FSH), androgen (DHEA, DHEAS, TT, FT), cortisol or sex hormone binding globulin (SHBG) levels.

TABLE 1
Patient Demographics, FSFI mean score range, baseline hormone levels

| | Completed Study | | Withdrew from study | | |
|---|---|---|---|---|---|
| | N = 50 | | N = 37 | | |
| Patient Demographics | | | | | |
| Age (years) | 41.1 ± 4.2 | | 40.3 ± 4.1 | | 0.4675 |
| BMI (kg/m²) | 24.4 ± 6.1 | | 24.9 ± 4.7 | | 0.7232 |
| Married | 43 | (86%) | 26 | (74.3%) | 0.1120 |
| Parous | 21 | (42%) | 13 | (36.1%) | 0.4803 |
| Race | | | | | 0.7718 |
| White | 25 | (50%) | 19 | (54.3%) | |
| Black | 8 | (16.0%) | 7 | (20.0%) | |
| Hispanic | 7 | (14.0%) | 4 | (11.4%) | |
| Asian | 7 | (14.0%) | 4 | (11.4%) | |
| FSFI mean score (range) | 27.2 | (2.8-34.8) | 27.0 | (2-36) | 0.8798 |
| Desire | 3.5 | (1.2-6) | 3.6 | (1.2-6) | 0.7046 |
| Arousal | 4.3 | (0-6) | 4.3 | (0-6) | 0.9449 |
| Lubrication | 4.8 | (0-6) | 4.9 | (0-6) | 0.6385 |
| Orgasm | 4.7 | (0-6) | 4.5 | (0-6) | 0.5845 |
| Satisfaction | 4.9 | (1.2-6) | 4.6 | (0.8-6) | 0.2974 |
| Pain | 5.1 | (0-6) | 5.2 | (0-6) | 0.6384 |
| Baseline Hormone levels | | | | | |

| | | | |
|---|---|---|---|
| AMH (ng/mL) | 1.0 ± 1.3 | 0.9 ± 1.4 | 0.9120 |
| FSH (mIU/mL) | 10.3 ± 5.4 | 14.3 ± 9.9 | 0.0419 |
| Total Testosterone (ng/dL) | 23.3 ± 14.0 | 21.8 ± 13.9 | 0.6351 |
| Free Testosterone (pg/mL) | 1.7 ± 1.2 | 1.7 ± 1.2 | 0.9737 |
| DHEA (ng/dL) | 247.7 ± 150.5 | 237.8 ± 118.5 | 0.7510 |
| DHEAS (ug/dL) | 141.1 ± 94.6 | 158.6 ± 93.0 | 0.4085 |
| SHBG (nmol/L) | 92.7 ± 43.2 | 90.2 ± 51.7 | 0.8146 |
| Cortisol (ug/dL) | 9.1 ± 5.0 | 9.2 ± 6.3 | 0.9405 |

Table 2 shows that following supplementation with DHEA all serum androgen levels (DHEA, DHEAS, TT, FT) increased from baseline (P<0.0001 for all); while FSH levels decreased from a baseline of 10.3±5.4 mIU/mL by 2.6±4.4 mIU/mL (P=0.009). SHBG decreased from a baseline of 92.7±43.2 nmol/L by 24.0±39.4 nmol/L (P=0.0002) There was no change noted in cortisol and AM11 levels.

Data presented in Table 2 also demonstrate that, following supplementation with DHEA, the FSFI score for the study group increased by 7% (from 27.2±6.9 to 29.2±5.6; P=0.0166). Domain scores for desire significantly increased by 17% and by 12% for arousal. Domain score for lubrication demonstrated an 8% trend towards improvement (P=0.0551), while no significant changes in domain scores for orgasm, satisfaction or pain were observed.

Women in the lowest starting FSFI score quartile (<25.7) revealed a 6.1±8.0 (34%) increase in the total FSFI score. The improvement for these women was noted in all domain categories, including desire 1.0±0.8 (40%), arousal 1.3±1.7 (46%), lubrication 0.8±2.3 (33%), orgasm 1.0±2.3 (54%), satisfaction 0.9±1.3 (24%), and pain 0.9±2.3 (25%). Moreover, FT increased more in patients with lowest starting FSFI score quartile (1.8±1.3 pg/mL) than among patients in highest quartile (≥31.9) (0.5±1.4 pg/mL, P=0.04).

TABLE 2
Effect of DHEA supplementation on sexual function and hormone
levels in a group of 50 premenopausal infertile women.

| | Mean | Std Dev | P-value |
|---|---|---|---|
| Δ FSFI Score | 2.0 | 5.6 | 0.0166 |
| Δ Desire | 0.6 | 1.1 | 0.0004 |
| Frequency | 0.5 | 1.1 | 0.0015 |
| Level | 0.5 | 1.0 | 0.0016 |
| Δ Arousal | 0.5 | 1.3 | 0.0122 |
| Frequency | 0.3 | 1.2 | 0.0917 |
| Level | 0.4 | 1.3 | 0.0232 |
| Confidence | 0.4 | 1.1 | 0.0124 |
| Satisfaction | 0.4 | 1.4 | 0.0443 |
| Δ Lubrication | 0.4 | 1.3 | 0.0551 |
| Frequency | 0.3 | 1.2 | 0.0517 |
| Difficulty | 0.3 | 1.4 | 0.1372 |
| Maintaining | 0.3 | 1.3 | 0.0844 |
| Difficulty | 0.2 | 1.5 | 0.3141 |
| Δ Orgasm | 0.1 | 1.5 | 0.6749 |
| Frequency | 0.0 | 1.4 | 0.9178 |
| Difficulty | 0.1 | 1.5 | 0.5581 |
| Satisfaction | 0.1 | 1.7 | 0.7298 |
| Δ Satisfaction | 0.3 | 1.2 | 0.1065 |
| Closeness | 0.3 | 1.1 | 0.1289 |
| Relationship | 0.2 | 1.2 | 0.1534 |
| Overall | 0.3 | 1.2 | 0.079 |
| Δ Pain | 0.2 | 1.3 | 0.2017 |
| During | 0.3 | 1.1 | 0.0428 |
| Following | 0.2 | 1.2 | 0.1847 |
| Level | 0.0 | 1.3 | 0.8235 |
| Δ Hormone | | | |
| AMH (ng/mL) | 0.0 | 0.6 | 0.7384 |
| FSH (mIU/mL) | −2.6 | 4.4 | 0.0092 |
| Total Testosterone (ng/dL) | 15.1 | 20.1 | <.0001 |
| Free Testosterone (pg/mL) | 1.4 | 2.2 | <.0001 |
| DHEA (ng/dL) | 235.1 | 189.2 | <.0001 |
| DHEAS (ug/dL) | 276.3 | 179.5 | <.0001 |
| SHBG (nmol/L) | −24.0 | 39.4 | 0.0002 |
| Cortisol (ug/dL) | 1.3 | 8.0 | 0.4527 |

TABLE 3
Effect of DHEA supplementation on sexual function and hormone levels
of premenopausal infertile women in the lowest starting FSFI score
quartile compared to those in the highest FSFI score quartile.

| | Baseline | Baseline | |
|---|---|---|---|
| | FSFI | FSFI | |
| | Score < 25.7 | Score ≥ 31.9 | |
| | N = 12 | N = 13 | P value |
| Baseline Characteristics | | | |
| Age (years) | 41.8 ± 4.0 | 41.6 ± 3.4 | 0.8828 |
| BMI (kg/m²) | 23.2 ± 2.6 | 23.5 ± 8.8 | 0.8977 |
| AMH (ng/mL) | 0.9 ± 1.2 | 1.0 ± 1.3 | 0.8656 |
| FSH (mIU/mL) | 9.7 ± 4.8 | 9.8 ± 5.3 | 0.9845 |
| Total Testosterone (ng/dL) | 27.4 ± 24.0 | 21.2 ± 9.5 | 0.3991 |
| Free Testosterone (pg/mL) | 1.3 ± 0.8 | 1.8 ± 1.6 | 0.4193 |
| DHEA (ng/dL) | 296.5 ± 184.5 | 221.8 ± 142.2 | 0.2842 |
| DHEAS (ug/dL) | 169.9 ± 151.1 | 140.1 ± 90.9 | 0.5686 |
| SHBG (nmol/L) | 69.8 ± 18.8 | 101.9 ± 50.4 | 0.0593 |
| Cortisol (ug/dL) | 8.4 ± 3.1 | 8.0 ± 3.8 | 0.7937 |
| Δ FSFI Score | 6.1 ± 8.0 | −1.0 ± 2.7 | 0.0059 |
| Δ Desire | 1.0 ± 0.8 | −0.1 ± 0.6 | 0.0003 |
| Δ Arousal | 1.3 ± 1.7 | −0.2 ± 0.5 | 0.0068 |
| Δ Lubrication | 0.8 ± 2.3 | −0.1 ± 0.6 | 0.1742 |
| Δ Orgasm | 1.0 ± 2.3 | −0.5 ± 1.1 | 0.0525 |
| Δ Satisfaction | 0.9 ± 1.3 | 0.0 ± 0.7 | 0.0422 |
| Δ Pain | 0.9 ± 2.3 | −0.1 ± 0.4 | 0.1292 |
| Δ Hormone | | | |
| AMH (ng/mL) | 0.0 ± 0.6 | 0.1 ± 0.6 | 0.9018 |
| FSH (mIU/mL) | −3.6 ± 2.6 | −1.6 ± 3.5 | 0.3866 |
| Total Testosterone (ng/dL) | 16.4 ± 18.5 | 11.8 ± 18.6 | 0.5504 |
| Free Testosterone (pg/mL) | 1.8 ± 1.3 | 0.5 ± 1.4 | 0.0359 |
| DHEA (ng/dL) | 291.0 ± 227.7 | 275.7 ± 186.6 | 0.8610 |
| DHEAS (ug/dL) | 305.6 ± 202.5 | 274.8 ± 131.0 | 0.6674 |
| SHBG (nmol/L) | −15.8 ± 41.1 | −21.0 ± 51.6 | 0.7912 |
| Cortisol (ug/dL) | 2.5 ± 1.6 | −0.4 ± 5.5 | 0.3461 |

The drawing and Table 3 show that the treatment effect of DHEA was primarily seen in women with low baseline FSFI scores. This shows that DHEA supplementation seems to help improve sexual function in women with low baseline sexual function rather than those with normal baseline function. That is, DHEA helps women with low baseline FSFI scores but not women with normal/high scores. Table 3 indicates that it was women with baseline FSFI Scores <25.7 who benefited the most from DHEA.

Effectiveness: Women with low baseline FSFI scores experienced a 6.1±8.0 (34%) increase in total FSFI score. Improvements in domain categories were noted for desire (40%), arousal (46%), lubrication (33%), orgasm (54%), satisfaction (24%), and pain (25%). The FSFI score for the whole study group increased by 7% (from 27.2±6.9 to 29.2±5.6; P=0.0166). Domain scores for desire increased by 17% (P=0.0004) and by 12% for arousal (P=0.0122); lubrication demonstrated an 8% trend towards improvement (P=0.0551), while no changes in domain scores for orgasm, satisfaction or pain were observed.

Because infertile women are usually relatively hypoandrogenic, 11, 22, 32, 33 and because, at CHR such patients are routinely supplemented with DHEA while their androgen levels are monitored, this patient population is ideal for assessing DHEA effects on female sexual function. This study was, indeed, conceived after many patients on DHEA supplementation reported improvements in libido and other sexual function parameters.

Because sexual dysfunction has been reported to be increased in infertile women,^{29, 30} we were not surprised that baseline FSFI scores (27.2±6.9, range 2.8-34.8) were only marginally lower than those previously described among healthy controls of similar age (30.5±5.3), but remarkably higher than among patients with female sexual arousal disorder (19.2±6.6).³¹ There was, however, no correlation noted between FSFI scores and patient demographics, ovarian reserve parameters or baseline androgen levels for the whole study group.

Also unsurprising were here observed changes in endocrine parameters following DHEA supplementation. The observed increases in all androgens, decrease in SHBG and FSH levels, and no change in AMH and cortisol levels are consistent with prior studies in menopausal women.³⁴

The whole patient cohort experienced improvements in sexual function. Considering that here investigated patients were primarily not F SD patients, this is quite a remarkable result, suggesting beneficial effects of DHEA supplementation at this age on female sexual wellbeing. Again unsurprising, patients in the lowest starting FSFI quartile experienced the most profound increase in scores. In other words, those study subjects who had most profound evidence of sexual dysfunction benefitted most from DHEA supplementation.

These patients also demonstrated a significantly larger increase in FT levels than patients in the highest FSFI quartile who did not experience improvements in FSFI scores. These data also suggest a dose-response relationship in patients with sexual dysfunction and absence of such a relationship in women with relatively normal sexual function.

While this makes biological sense, androgen metabolism is complex: Steroidogenic capacity declines with age and multiple genetic and enzymatic factors affect conversion of DHEA to bioactive testosterone.³² This may also be one reason why some prior studies of DHEA supplementation in women with sexual dysfunction have been unsuccessful¹⁴; although some improvements in peri- and post-menopausal women¹⁸ and those suffering from adrenal insufficiency¹⁹ have been described.

Another obvious reason is that, like in female infertility, not every woman with sexual dysfunction is hypoandrogenic. One cannot expect DHEA to be effective in normo-androgenic patients. Primarily hypoandrogenic premenopausal women with sexual dysfunction will, therefore, likely respond well to DHEA supplementation. This raises the question how a female patient in this age group can be defined as hypoandrogenic since there are no agreed to testosterone levels below which a woman can be currently classified a being hypoandrogenic.¹⁴ In infertility practice, we consider women as androgen-deficient if their testosterone levels are in the lower third of normal range and if they demonstrate sex hormone binding globulin levels above 100 nmol/L.^{11, 22, 32} Whether this also represents an appropriate definition for hypoandrogenic women with sexual dysfunction in a general population remains to be confirmed.

Several limitations in the design of this study warrant caution: First, we reemphasize that here presented study is not a study of women diagnosed with FSD but of premenopausal women with infertility. They, therefore, are more representative of a general population, with possibly only mildly higher prevalence of sexual dysfunction.^{29, 30}

Patients in this observational study acted as their own controls in that FSFI scores in each patient were compared before and after DHEA supplementation. This study format is inferior to that of a prospectively randomized placebo-controlled trial but, still, offers an acceptable level of evidence, especially considering that involved patients did not have primary personal interests in the issue here investigated, as none had entered treatment because of sexual dysfunction or with the intent of improving her sexual function.

Finally, 42.5% of women did not complete the second questionnaire. Such a large dropout rate raises the possibility of selection bias. Not able to refute such a possibility, we would argue that the most likely drop outs would be those women least affected by DHEA, while those most beneficially affected would want to stay in the study. It, therefore, is possible that here presented data somewhat exaggerate the beneficial effects of DHEA in women with sexual dysfunction, even though, as Table 1 demonstrates, drop outs and study patients were very similar in their respective clinical phenotypes. This would also explain the remarkably clear and statistically robust outcome improvements observed in a relatively small patient population.

The observational study offers evidence that DHEA supplementation improves sexual function in premenopausal women with low baseline FSFI scores.

Effectiveness: Women with low baseline FSFI scores experienced a 6.1±8.0 (34%) increase in total FSFI score. Improvements in domain categories were noted for desire (40%), arousal (46%), lubrication (33%), orgasm (54%), satisfaction (24%), and pain (25%). The FSFI score for the whole study group increased by 7% (from 27.2±6.9 to 29.2±5.6; P=0.0166). Domain scores for desire increased by 17% (P=0.0004) and by 12% for arousal (P=0.0122); lubrication demonstrated an 8% trend towards improvement (P=0.0551), while no changes in domain scores for orgasm, satisfaction or pain were observed.

The measurement procedures for measuring serum androgen and FSH are conventional. The formulation for an oral dosage of DHEA is conventional.

Before a premenopausal woman should be prescribed, administered or provided or otherwise delivered DHEA dosages of 25 mg, an evaluation of her FSFI score should be done to make sure the baseline FSFI score is no more than 25.7. The premenopausal woman could perform the evaluation herself if given the questionnaire and calculation sheet found in the FSFI scoring appendix. The premenopausal woman could then obtain DHEA supplements on her own. A responsible DHEA provider should provide some guidance as to what the premenopausal woman should expect based on her baseline FSFI score with respect to increasing sexual function (or sex drive) over a period of time such as 4-6 weeks of daily taking of the DHEA dosages of 25 mg once, twice, thrice or four times daily. If such a responsible DHEA provider is informed as to the reason that the premenopausal woman wants the DHEA, the responsible provider should only recommend taking the DHEA if her FSFI score is some value less than 25.7. If such criteria are met, such as where the premenopausal woman confirms her baseline FSFI score is sufficiently low, then the responsible provider can deliver the DHEA supplements accordingly.

It is known to use a conventional Female Sexual Distress Scale (FSDS) to diagnose a patient with Female Sexual Dysfunction (F SD) by documenting distress. By analogy, the reliance on an FSFI score in accordance with the invention could be replaced by reliance on a comparable FSDS score instead for which a score greater than or equal to 11, which effectively discriminates between women with FSD and those with no FSD, can likewise form the basis for determining whether to administer DHEA supplementation. That is, if the FSDS score is greater than or equal to 11, then the premenopausal woman who has FSD should take DHEA supplementation to improve her sexual function or sex drive. The FSDS-R is provided herein following the FSFI Domain Scores and Full Scale Score.

One purpose of DHEA supplementation is raising testosterone levels. In other words, the biologically active hormone is testosterone. Its application, however, has more side effects. But some patients have a hard time converting DHEA to testosterone [Shohat-Tal A, Sen A, Barad D H, Kushnir V, Gleicher N. Genetics of androgen metabolism in women with infertility and hypoandrogenism. Nat Rev Endocrinol 2015; 11(7):429-441] and, therefore, must be treated directly with testosterone. The testosterone dosage is 0.05 mg transdermal by pump between once to 2-times per day for the same time period that DHEA supplementation dosages are taken. The peripheral testosterone levels and SHBG in blood are monitored and adjusted accordingly. Sex hormone binding globulin (SHBG) is a protein that is produced by the liver and binds tightly to the hormones testosterone, dihydrotestosterone (DHT), and estradiol (an estrogen). If patients require testosterone directly, the DHEA supplementation is still continued. Our experience with DHEA treatments of infertile patients is that those few (and that is less than 5% of patients overall, and a little more among African-descent women) who do not raise their testosterone levels in response to DHEA supplementation, very similar testosterone levels are achieved by giving them 0.05 mg transdermal by pump between once to 2-times per day for the same time period.

Although the study pertained to the taking of DHEA supplementation by a premenopausal woman for four to six weeks, the treatment could be continued for many months or even years if the woman continues to have improved sexual function.

While the foregoing description and drawings represent the preferred embodiments of the present invention, it will be understood that various changes and modifications may be made without departing from the scope of the present invention.

### BIBLIOGRAPHY

1. McCool M E, Zuelke A, Theurich M A, Knuettel H, Ricci C, Apfelbacher C.

Prevalence of Female Sexual Dysfunction Among Premenopausal Women: A Systematic Review and Meta-Analysis of Observational Studies. Sexual medicine reviews 2016; 4(3):197-212. doi:10.1016/j.sxmr.2016.03.002.
2. Weinberger J M, Houman J, Caron A T, Anger J. Female sexual dysfunction: A systematic review of outcomes across various treatment modalities. Sexual medicine reviews 2018. doi:10.1016/j.sxmr.2017.12.004.
3. Joffe H V, Chang C, Sewell C, et al. FDA Approval of Flibanserin—Treating Hypoactive Sexual Desire Disorder. N Engl J Med 2016; 374(2):101-104. doi:10.1056/NEJMp 1513686.
4. Basson R, Berman J, Burnett A, et al. Report of the international consensus development conference on female sexual dysfunction: definitions and classifications. J Urol 2000; 163(3):888-893.
5. Guay A T, Jacobson J. Decreased free testosterone and dehydroepiandrosterone-sulfate (DHEA-S) levels in women with decreased libido. J Sex Marital Ther 2002; 28 Suppl 1:129-142. doi:10.1080/00926230252851258.
6. Turna B, Apaydin E, Semerci B, Altay B, Cikili N, Nazli O. Women with low libido: correlation of decreased androgen levels with female sexual function index. Int J Impot Res 2005; 17(2):148-153. doi:10.1038/sj.ijir.3901294.
7. Nappi R E. To be or not to be in sexual desire: the androgen dilemma. Climacteric 2015;18(5):672-674. doi:10.3109/13697137.2015.1064268.
8. Worsley R, Bell R J, Gartoulla P, Davis S R. Prevalence and Predictors of Low Sexual Desire, Sexually Related Personal Distress, and Hypoactive Sexual Desire Dysfunction in a Community-Based Sample of Midlife Women. J Sex Med 2017; 14(5):675-686. doi: 10.1016/j.j sxm.2017.03.254.
9. Kushnir M M, Blamires T, Rockwood A L, et al. Liquid chromatography-tandem mass spectrometry assay for androstenedione, dehydroepiandrosterone, and testosterone with pediatric and adult reference intervals. Clin Chem 2010; 56(7):1138-1147. doi:10.1373/clinchem.2010.143222.
10. Orentreich N, Brind J L, Rizer R L, Vogelman J H. Age changes and sex differences in serum dehydroepiandrosterone sulfate concentrations throughout adulthood. J Clin Endocrinol Metab 1984; 59(3):551-555. doi:10.1210/jcem-59-3-551.
11. Gleicher N, Kim A, Weghofer A, et al. Hypoandrogenism in association with diminished functional ovarian reserve. Hum Reprod 2013; 28(4):1084-1091. doi:10.1093/humrep/det033.
12. Gleicher N, Kushnir V A, Weghofer A, Barad D H. The importance of adrenal hypoandrogenism in infertile women with low functional ovarian reserve: a case study of associated adrenal insufficiency. Reprod Biol Endocrinol 2016; 14(1):23. doi:10.1186/s12958-016-0158-9.
13. Robinson P J, Bell R J, Christakis M K, Ivezic S R, Davis S R. Aromatase inhibitors are associated with low sexual desire causing distress and fecal incontinence in women: an observational study. J Sex Med 2017; 14(12):1566-1574. doi:10.1016/j.jsxm.2017.09.018.
14. Davis S R, Worsley R, Miller K K, Parish S J, Santoro N. Androgens and Female Sexual Function and Dysfunction—Findings From the Fourth International Consultation of Sexual Medicine. J Sex Med 2016; 13(2):168-178. doi:10.1016/j.jsxm.2015.12.033.
15. Khera M. Testosterone therapy for female sexual dysfunction. Sexual medicine reviews 2015; 3(3):137-144. doi:10.1002/smrj.53.
16. Panjari M, Davis S R. DHEA therapy for women: effect on sexual function and wellbeing. Hum Reprod Update 2007; 13(3):239-248. doi:10.1093/humupd/dm1055.
17. Spark R F. Dehydroepiandrosterone: a springboard hormone for female sexuality. Fertil Steril 2002; 77 Suppl 4:S19-25. doi:10.1016/S0015-0282(02)02987-4.
18. Scheffers C S, Armstrong S, Cantineau A E, Farquhar C, Jordan V. Dehydroepiandrosterone for women in the peri- or postmenopausal phase. Cochrane Database Syst Rev 2015; 1:CD011066. doi:10.1002/14651858.CD011066.pub2.
19. Arlt W, Callies F, van Vlijmen J C, et al. Dehydroepiandrosterone replacement in women with adrenal insufficiency. N Engl J Med 1999; 341(14):1013-1020. doi:10.1056/NEJM199909303411401.
20. Labrie F, Archer D F, Koltun W, et al. Efficacy of intravaginal dehydroepiandrosterone (DHEA) on moderate to severe dyspareunia and vaginal dryness, symptoms of vulvovaginal atrophy, and of the genitourinary syndrome of menopause. Menopause 2016; 23(3):243-256. doi:10.1097/GME.0000000000000571.
21. Gleicher N, Barad D H. Dehydroepiandrosterone (DHEA) supplementation in diminished ovarian reserve (DOR). Reprod Biol Endocrinol 2011; 9:67. doi:10.1186/1477-7827-9-67.
22. Gleicher N, Kim A, Weghofer A, et al. Starting and resulting testosterone levels after androgen supplementation determine at all ages in vitro fertilization (IVF) pregnancy rates in women with diminished ovarian reserve (DOR). J Assist Reprod Genet 2013; 30(1):49-62. doi:10.1007/s10815-012-9890-z.
23. Gleicher N, Weghofer A, Barad D H. Improvement in diminished ovarian reserve after dehydroepiandrosterone supplementation. Reprod Biomed Online 2010; 21(3):360-365. doi:10.1016/j.rbmo.2010.04.006.
24. Barad D, Brill H, Gleicher N. Update on the use of dehydroepiandrosterone supplementation among women with diminished ovarian function. J Assist Reprod Genet 2007; 24(12):629-634. doi:10.1007/s10815-007-9178-x.
25. Sen A, Hammes S R. Granulosa cell-specific androgen receptors are critical regulators of ovarian development and function. Mot Endocrinol 2010; 24(7):1393-1403. doi:10.1210/me.2010-0006.
26. Sen A, Prizant H, Light A, et al. Androgens regulate ovarian follicular development by increasing follicle stimulating hormone receptor and microRNA-125b expression. Proc Natl Acad Sci USA 2014; 111(8):3008-3013. doi:10.1073/pnas.1318978111.
27. Lin L T, Wang P H, Chen S N, et al. Protection of cumulus cells following dehydroepiandrosterone supplementation. Gynecol Endocrinol 2017; 33(2):100-104. doi:10.1080/09513590.2016.1214262.
28. Prizant H, Gleicher N, Sen A. Androgen actions in the ovary: balance is key. J Endocrinol 2014; 222(3):R141-51. doi:10.1530/JOE-14-0296.
29. Mendonça C R, Arruda J T, Noll M, Campoli PMO, Amaral WND. Sexual dysfunction in infertile women: A systematic review and meta-analysis. Eur J Obstet Gynecol Reprod Biol 2017; 215:153-163. doi:10.1016/j.ejogrb.2017.06.013.
30. Millheiser L S, Helmer A E, Quintero R B, Westphal L M, Milki A A, Lathi R B. Is infertility a risk factor for female sexual dysfunction? A case-control study. Fertil Steril 2010; 94(6):2022-2025. doi:10.1016/j.fertnstert.2010.01.037.
31. Rosen R, Brown C, Heiman J, et al. The Female Sexual Function Index (FSFI): a multidimensional self-report instrument for the assessment of female sexual function. J Sex Marital Ther 2000; 26(2):191-208. doi:10.1080/009262300278597.
32. Shohat-Tal A, Sen A, Barad D H, Kushnir V, Gleicher N. Genetics of androgen metabolism in women with infertility and hypoandrogenism. Nat Rev Endocrinol 2015; 11(7):429-441. doi:10.1038/nrendo.2015.64.
33. Gleicher N, Seier K, Kushnir V A, et al. Associations between peripheral androgens and cortisol in infertile women. J Steroid Biochem Mol Biol 2016. doi:10.1016/j.jsbmb.2016.01.004.
34. Stomati M, Monteleone P, Casarosa E, et al. Six-month oral dehydroepiandrosterone supplementation in early and late postmenopause. Gynecol Endocrinol 2000; 14(5):342-363. doi:10.3109/09513590009167703.

### FSFI Domain Scores and Full Scale Score

The individual domain scores and full scale (overall) score of the FSFI can be derived from the computational formula outlined in the table below. For individual domain scores, add the scores of the individual items that comprise the domain and multiply the sum by the domain factor (see below). Add the six domain scores to obtain the full scale score. It should be noted that within the individual domains, a domain score of zero indicates that the subject reported having no sexual activity during the past month. Subject scores can be entered in the right-hand colum.

| | | | | Mini- | Maxi- | |
|---|---|---|---|---|---|---|
| | | Score | | mum | mum | |
| Domain | Questions | Range | Factor | Score | Score | Score |
| Desire | 1, 2 | 1-5 | 0.6 | 1.2 | 6.0 | |
| Arousal | 3, 4, 5, 6 | 0-5 | 0.3 | 0 | 6.0 | |
| Lubrication | 7, 8, 9, 10 | 0-5 | 0.3 | 0 | 6.0 | |
| Orgasm | 11, 12, 13 | 0-5 | 0.4 | 0 | 6.0 | |
| Satisfaction | 14, 15, 16 | 0 (or 1)-5 | 0.4 | 0.8 | 6.0 | |
| Pain | 17, 18, 19 | 0-5 | 0.4 | 0 | 6.0 | |

| | | |
|---|---|---|
| Full Scale Score Range | 2.0 | 36.0 |

The Female Sexual Distress Scale-Revised (FSDS_R; revised 2005): Screening Questionnaire for Measuring Sexually Related Personal Distress in Women With Female Sexual Dysfunction (FSD) Name: Date:

Below is a list of feelings and problems that women sometimes have concerning their sexuality. Please read each item carefully, and circle the number that best describes HOW OFTEN THAT PROBLEM HAS BOTHERED YOU OR CAUSED YOU DISTRESS DURING THE PAST 30 DAYS INCLUDING TODAY. Circle only one number for each item, and take care not to skip any items. If you change your mind, erase your first circle carefully. Read the example before beginning, and if you have any questions please ask about them.

Example: How often did you feel: Personal responsibility for your sexual problems.

| NEVER | RARELY | OCCASIONALLY | FREQUENTLY | ALWAYS |
|---|---|---|---|---|
| 0 | 1 | 2 | 3 | 4 |

How often did you feel

| | | | | | | |
|---|---|---|---|---|---|---|
| 1. | Distressed about your sex life | 0 | 1 | 2 | 3 | 4 |
| 2. | Unhappy about your sexual relationship | 0 | 1 | 2 | 3 | 4 |
| 3. | Guilty about sexual difficulties | 0 | 1 | 2 | 3 | 4 |
| 4. | Frustrated by your sexual problems | 0 | 1 | 2 | 3 | 4 |
| 5. | Stressed about sex | 0 | 1 | 2 | 3 | 4 |
| 6. | Inferior because of sexual problems | 0 | 1 | 2 | 3 | 4 |
| 7. | Worried about sex | 0 | 1 | 2 | 3 | 4 |
| 8. | Sexually inadequate | 0 | 1 | 2 | 3 | 4 |
| 9. | Regrets about your sexuality | 0 | 1 | 2 | 3 | 4 |
| 10. | Embarrassed about sexual problems | 0 | 1 | 2 | 3 | 4 |
| 11. | Dissatisfied with your sex life | 0 | 1 | 2 | 3 | 4 |
| 12. | Angry about your sex life | 0 | 1 | 2 | 3 | 4 |
| 13. | Bothered by low sexual desire | 0 | 1 | 2 | 3 | 4 |

| | |
|---|---|
| A score ≥ 11 effectively discriminates between women with FSD and FSD.* | Total [ ] |

Copyright ©2000 by American Foundation for Urological Disease Inc.
*DeRogatis L, et al. J Sex Med. 2008; 5: 357-364

The Female Sexual Distress Scale-Revised (FSDS_R; revised 2005): Screening Questionnaire for Measuring Sexually Related Personal Distress in Women With Female Sexual Dysfunction (FSD)

The presence of personal distress is central to the diagnosis of hypoactive sexual desire disorder (HSDD). This is recognized in the DSM-IV®-TR and other recent diagnostic guidelines for female sexual dysfunction (FSD), including those emanating from the 1999 International Consensus Development Conference on FSD, which stated that women with decreased sexual desire can only be diagnosed with HSDD if they have evidence of associated personal distress.¹

Accordingly, the Female Sexual Distress Scale (FSDS) was developed to provide a standardize, quantitative measure of sexually related personal distress in women.²

The FSDS-R differs from the FSDS in that it includes one additional question that asks women to rate distress related to low sexual desire, consistent with its use as part of the diagnostic algorithm for HSDD.¹

A copy of the FSDS-R appears on the reverse side of this card.

1. DeRogatis L, et al. J Sex Med. 2008; 5:357-364.

2. DeRogatis L, et al. J Sex Marital Ther. 2002; 28:317-330.

Copyright ©2010, Boehringer Ingelheim Pharmaceuticals, Inc.

What is claimed is:

1. A method of improving female sexual function through dehydroepiandrosterone (DHEA) supplementation, comprising the steps of:
giving a premenopausal woman access to information for her to provide input necessary for computing a Female Sexual Function Index (FSFI) baseline;
confirming that the computed FSFI baseline of the premenopausal woman is less than or equal to 25.7 derived from the input; and
supplying DHEA supplementation to the premenopausal woman to allow the premenopausal woman to consume the DHEA supplementation in 25 mg micronized dosages daily over a period of time of four to six weeks to improve the female sexual function.

2. The method of claim 1, further comprising:
increasing the FSFI baseline of the premenopausal woman by at least seven percent as a result of her consumption over the period of time of the DHEA supplementation supplied, the increasing of the FSFI baseline constituting an improvement in the female sexual function.

3. The method of claim 1, further comprising:
increasing a serum androgen level of the premenopausal woman over the period of time and decreasing a follicle-stimulating hormone (FSH) level of the premenopausal woman over the period of time, the increasing in the serum androgen level and the decreasing in the FSH level both arising as a result of the premenopausal woman consuming the DHEA supplementation supplied over the period of time and constituting an improvement in the female sexual function.

4. The method of claim 1, wherein the DHEA supplementation supplied is in 100 mg sizes, but splitting the DHEA supplementation into four results in realizing the micronized dosages of 25 mg each.

5. The method of claim 1, wherein at least one of six domain scores of the premenopausal woman increases after the DHEA supplementation is taken daily for the period of time, wherein the six domains are desire, arousal, lubrication, orgasm, satisfaction, and pain and an increase in any one of the sex domain scores over the period of time constitutes an improvement in the female sexual function.

6. The method of claim 5, wherein at least one of the six domain scores increase in accord with at least an increase of 40%, 46%, 33%, 54%, 24% and 25% respectively, corresponding to that for the desire, the arousal, the lubrication, the orgasm, the satisfaction, and the pain.

7. The method of claim 1, further comprising:
calculating the FSFI baseline based upon the input from the premenopausal woman that reflects her answers to queries that are part of the information provided.

8. The method of claim 1, wherein the information is provided online and the FSFI baseline is calculated online.

9. A method of improving female sexual function through dehydroepiandrosterone (DHEA) supplementation, comprising the steps of:
confirming that a Female Sexual Function Index (FSFI) baseline of a premenopausal woman is no more than 25.7 or that a Female Sexual Distress Scale-Revised (FSDS-R) baseline is no less than 11; and
prescribing, administering or giving to the premenopausal woman micronized oral dosages of 25 mg of DHEA supplement and repeating as necessary to that the premenopausal woman swallows the micronized oral dosages daily for a period of time of four to six weeks and which results in:
an increase in a serum androgen level of the premenopausal woman over a period of time as a consequence of the premenopausal woman taking or being administered the micronized oral dosages of the DHEA supplement daily over the period of time; and
a decrease in follicle-stimulating hormone (FSH) level of the premenopausal woman over the period of time as a consequence of the premenopausal woman taking or being administered the micronized oral dosages of the DHEA supplement daily over the period of time,
wherein the increase in the serum androgen level and decrease in the FSH level improve the female sexual function of the premenopausal woman.

10. The method of claim 9, further comprising the steps of:
confirming that the serum androgen level increased by having measurements made of a serum androgen level of the premenopausal woman before and after the DHEA supplement is taken daily for the period of time and then making a comparison of the measurements of the androgen level before and after; and
confirming that the follicle-stimulating hormone (FSH) level decreased by having measurements made of the FSH level of the premenopausal woman before and after the DHEA supplement is taken daily for the period of time and then making a comparison of the measurements of the FSH level before and after.

11. The method of claim 9, wherein the micronized oral dosage is 100 mg, further comprising the step of:
splitting the micronized oral dosage of 100 mg into 25 mg dosages, which are taken once, twice, thrice or four times daily.

12. The method of claim 9, further comprising the step of:
determining that the FSFI baseline increased by at least seven percent after the DHEA is taken daily for the period of time.

13. The method of claim 9, further comprising the step of:
determining that domain scores increased percent after the DHEA is taken daily for the period of time.

14. The method of claim 9, further comprising the step of:
determining that at least one of a plurality of the scores of domains selected from the group consisting of desire, arousal, lubrication, orgasm, satisfaction, and pain increase at least by 40%, 46%, 33%, 54%, 24% and 25% respectively.

15. The method of claim 15, further comprising:
prescribing, administering or giving the premenopausal woman 0.05 mg transdermal by pump between once to 2-times per day for the period of time.

16. A method of improving female sexual function through dehydroepiandrosterone (DHEA) supplementation, comprising the steps of:
giving a premenopausal woman access to information for her to provide input necessary for computing a Female Sexual Distress Scale-Revised (FSDS-R) baseline;
confirming that the computed FSDS-R baseline of the premenopausal woman is equal to or greater than 11 derived from the input; and
supplying DHEA supplementation to the premenopausal woman to allow the premenopausal woman to consume the DHEA supplementation in 25 mg micronized dosages daily over a period of time of four to six weeks to improve the female sexual function.

17. The method of claim 16, further comprising:
increasing the FSDS-R baseline of the premenopausal woman as a result of her consumption over the period of time of the DHEA supplementation supplied, the increasing of the FSDS-R baseline constituting an improvement in the female sexual function.

18. The method of claim 16, further comprising:
increasing a serum androgen level of the premenopausal woman over the period of time and decreasing a follicle-stimulating hormone (FSH) level of the premenopausal woman over the period of time, the increasing in the serum androgen level and the decreasing in the FSH level both arising as a result of the premenopausal woman consuming the DHEA supplementation supplied over the period of time and constituting an improvement in the female sexual function.

19. The method of claim 16, wherein the DHEA supplementation supplied is in 100 mg sizes, but splitting the DHEA supplementation into four results in realizing the micronized dosages of 25 mg each.

20. The method of claim 16, wherein at least one of six domain scores of the premenopausal woman increases after the DHEA supplementation is taken daily for the period of time, wherein the six domains are desire, arousal, lubrication, orgasm, satisfaction, and pain and an increase in any one of the sex domain scores over the period of time constitutes an improvement in the female sexual function.

21. The method of claim 16, wherein the information is provided online and the FSDS-R baseline is calculated online.
